(19) United States
(12) Patent Application Publication
(10) Pub. No.: US 2026/0153485 A1
(43) Pub. Date: Jun. 4, 2026

(54) METHOD FOR DETECTING HALOGENATED ORGANIC SUBSTANCES IN LANDFILL LEACHATE AND POLLUTED WATER THEREOF

(71) Applicant: CHINESE RESEARCH ACADEMY OF ENVIRONMENTAL SCIENCES, Beijing (CN)

(72) Inventors: Xiaosong HE, Beijing (CN); Haoqun SHA, Beijing (CN); Beidou XI, Beijing (CN); Zengyin LIANG, Beijing (CN); Jing ZHENG, Beijing (CN)

(73) Assignee: CHINESE RESEARCH ACADEMY OF ENVIRONMENTAL SCIENCES, Beijing (CN)

(21) Appl. No.: 18/986,825

(22) Filed: Dec. 19, 2024

(30) Foreign Application Priority Data
Dec. 3, 2024 (CN) 2024117661448

(51) Int. Cl. G01N 30/72; G01N 27/62; G01N 30/06; G01N 30/12; G01N 30/14; G01N 33/18

(52) U.S. Cl. CPC G01N 30/72; G01N 27/62; G01N 30/12; G01N 30/14; G01N 33/1826; G01N 2030/062; G01N 2030/126; G01N 2030/146; G01N 2223/345

(57) ABSTRACT

The present invention discloses a method for detecting halogenated organic substances in a landfill leachate and water bodies polluted with the leachate, by a combined solid phase extraction method comprising HLB solid phase extraction and PPL solid phase extraction, acidifying the sample using formic acid, and subjecting the aqueous sample to solid phase extraction enrichment using an HLB solid phase extraction column and a PPL solid phase extraction column, respectively, elution with methanol, the enriched samples are then subjected to FT-ICR MS detection, performing molecular formula matching of organic molecules, molecular screening, molecular information summarization and statistical analysis of data of samples separately on two FT-ICR MS detection data, collecting the two together gives all dissolvable halogenated organic compounds (HOCs) in samples of water, halo-organic species are then visualized by Sankey diagrams.

### CROSS-REFERENCE TO RELATED APPLICATIONS

This application claims priority to Chinese Patent Application Ser. No. CN2024117661448 filed on 3 Dec. 2024.

### FIELD OF THE INVENTION

The present invention relates to the field of environmental protection and in particular to a method for detecting halogenated organic substances in landfill leachate and polluted water bodies thereof.

### BACKGROUND

Halogenated organic compounds (HOCs) are also referred to as halogenated organic pollutants, which includes fluorinated organics, chlorinated organics, brominated organics, and the like. HOCs serious threats to the environment and human health for its persistence and accumulative capacity in organisms. Most HOCs are endocrine disruptors, neurotoxicity, immunotoxicity and reproductive damage to organisms. Household waste contains a wide variety of HOCs, as a result of rainfall, hydration and biodegradation of the waste itself, enrichment of the waste from the solid phase into the landfill leachate occurs. HOCs migrate with the leachate into the groundwater once the barrier layer of the landfill breaks, thereby creating a serious threat to the environment and surrounding organisms.

Mass spectrometry has become an effective method to detect HOCs in the environment, for example, high performance liquid chromatography coupled with electrospray ionization-mass spectrometry has been used to identify polar HOCs, and gas chromatography-mass spectrometry is used to detect low mass and volatile HOCs. However, these methods can only detect part of the HOCs in the environment, a large number of unknown HOCs remain undetected, therefore non-targeted ultra-high resolution mass spectrometry based techniques have been applied in recent years for HOCs detection. Detection of HOCs in high salt organic wastewater is performed as before using Orbitrap Mass Spectrometry (CN 118183937 A), but Orbitrap Mass Spectrometry typically has a resolution between 100,000 and 1 million, while Fourier Transform Ion Cyclotron Resonance Mass Spectrometry (FT-ICR MS) typically has a resolution above million, so FT-ICR MS has better resolution than Orbitrap Mass Spectrometry. In addition to resolution, FT-ICR MS has better precision than Orbitrap Mass Spectrometry in low mass ranges, so FT-ICR MS is an effective method for high-precision characterization of HOCs in environmental samples.

When HOCs are characterized in the environment using FT-ICR MS, the HOCs in the sample are typically enriched using a packed solid phase extraction column such as C18 or HLB during the solid phase extraction stage of sample pre-treatment. As solid phase extraction can only extract a fraction of the HOCs in the aqueous sample (enrichment of DOC around 50%), so the final resultant halo-dissolvable organic molecules are only a fraction of the original sample, which poses certain difficulties to fully elucidate the exfoliation, migration and conversion of HOCs in the environment.

### SUMMARY OF THE INVENTION

To solve the above problems, the present invention provides a combined solid phase extraction method comprising HLB solid phase extraction and PPL solid phase extraction, in which, samples of water are enriched by solid phase extraction using an HLB solid phase extraction column and a PPL solid phase extraction column, respectively, and then are detected by Fourier Transform Ion Cyclotron Resonance Mass Spectrometry (FT-ICR MS), respectively. The present invention is completed by performing molecular formula matching of organic molecules, molecular screening, molecular information summarization and/or statistical analysis of the data of samples separately on two data of FT-ICR MS, by combining the data to obtain all dissolvable halogenated organic substances in the samples of water, allowing for a more comprehensive collection of halogenated organic compounds in the water samples.

It is an object of the present invention to provide a method for detecting halogenated organic substances in landfill leachate and polluted water bodies thereof, by combined solid phase extraction method comprising HLB solid phase extraction and PPL solid phase extraction, the aqueous samples are enriched by solid phase extraction using an HLB solid phase extraction column and a PPL solid phase extraction column, respectively, and the enriched samples are then subjected to FT-ICR MS detection, and two detection data of FT-ICR MS are subjected to molecular formula matching of organic molecules, molecular screening, molecular information summarization, and/or statistical analysis of the data of samples, respectively, and the two are combined or merged to obtain all dissolvable halogenated organic substances in the samples of water.

In preferred embodiment, fresh leachate stocks solution collected from living landfills and samples of groundwater up and down near the landfills are transported to a laboratory within one week after the collection for preservation and pre-treatment.

In further preferred embodiment, collected aqueous sample is passed through a filter membrane, preferably 0.45 μm filter membrane, to sufficiently remove poorly dissolvable organic substances, such as suspended matters, and the concentration of dissolvable organic carbon is measured, and then the volume required for solid phase extraction is determined based on the concentration of DOC.

In the present invention, as a method for measuring the concentration of DOC, methods common in the art such as heat oxidation, ultraviolet irradiation-persulfate oxidation, OH radical oxidation and the like can be used. Typical DOC meter in the art can be employed, and this is not particularly limited.

The present inventors have found that FT-ICR MS detection is excellent for HLB and PPL solid phase extraction when the value of DOC is between 90-110 mg/L while the DOC adsorbed by the solid phase extraction columns is enriched to 1 mL after elution.

Accordingly, in a preferred embodiment, to ensure the comprehensiveness and completeness of the section and analysis results, the DOCs of the landfill leachate stock samples, and the upper and downstream groundwater samples are measured respectively after passing through the filter membrane. The samples of the leachate stock are diluted until the DOC was equal to the DOC of samples of groundwater. The inventors have found that the volume of the water sample required for solid phase extraction is best determined based on DOC enrichment of between 30% and 50% by the solid phase extraction columns.

Following pretreatment described above, aqueous sample is next subjected to solid phase extraction.

In a preferred embodiment, two samples of filtered water are taken respectively according to the above calculated volumes, and the pH of the samples is adjusted to a predetermined value with formic acid solution. An HLB solid phase extraction column and a PPL solid phase extraction column are mounted on a solid phase extraction device, respectively. Each solid phase extraction column is rinsed and activated sequentially with methanol and formic acid solution, and then the water samples are slowly added to the HLB solid phase extraction column and the PPL solid phase extraction column, respectively at certain flow rate, such as 5 mL min⁻¹, to enrich halogenated organic compounds. After the aqueous samples flow out, formic acid is added to wash the HLB solid phase extraction column and the PPL solid phase extraction column, respectively, to remove salts, and then the columns are dried by nitrogen blowing and are eluted with methanol separately. The eluents are collected and blown with nitrogen until methanol is fully volatilized, to yield enriched samples from HLB and PPL solid phase extraction columns, respectively, which will be subjected to subsequent FT-ICR MS detection.

In a more preferred embodiment, two filtered water samples are taken respectively according to the above calculated volumes. and the pH of samples is adjusted to 2.0 with 98% formic acid solution. The HLB solid phase extraction column and the PPL solid phase extraction column are mounted on the solid phase extraction device, respectively. Each solid phase extraction column is rinsed and activated sequentially with 12 mL methanol and 12 mL 0.01 mol/L formic acid solution, and then the aqueous sample are slowly added to the HLB solid phase extraction column and the PPL solid phase extraction column, respectively at a flow rate of 5 mL min⁻¹, to enrich the halogenated organic compounds. After the aqueous samples flow out, 18 mL 0.01 mol/L formic acid is added to wash the HLB and PPL solid phase extraction columns, respectively, to remove salts, and then the columns are dried by nitrogen blowing, and are eluted with 12 mL methanol, respectively. The eluents are collected and blown with nitrogen to complete volatilization of the methanol, to obtain enriched samples from HLB and PPL solid phase extraction columns, respectively.

In the present invention, lipophilic divinylbenzene and hydrophilic N-vinylpyrrolidone copolymer is used as packing materials of the HLB solid phase extraction column, and commonly used HLB solid phase extraction column, such as Oasis® HLB solid phase extraction column, can be used.

In the present invention, modified styrene-divinylbenzene polymer is used as packing materials of the PPL solid phase extraction column, and commonly used PPL solid phase extraction column, such as Bond Elut PPL solid phase extraction column, can be used.

Prior to FT-ICR MS detection, enriched samples from HLB and PPL solid-phase extraction columns are dissolved with methanol, and then the samples are detected with SolariX type FT-ICR MS from Bruker Company.

For FT-ICR MS detection data of enriched samples from HLB and PPL solid phase extraction columns, molecular formula matching, molecular screening, molecular information summarization are performed, and visual analysis of the data for halo-dissolvable organic substances in samples using Sankey diagrams to clearly demonstrate species of the halo-dissolvable organic substances.

In a preferred embodiment of the present invention, molecular formula matching of organic molecules is performed on two FT-ICR MS detection data, followed by molecular screening, molecular information summarization and visual analysis by Sankey diagrams, by dissolvable organic substances ultra-resolution mass spectrometry matching platform FTMSAnalysis developed based on a Gaussian distribution alignment algorithm.

The dissolvable organic substances ultra-high resolution mass spectrometry matching platform FTMSAnalysis platform (see: Fu Q-L, Fujii M, Ma R. Development of a Gaussian-Based Alignment Algorithm for the Ultrahigh-Resolution Mass Spectra of Dissolved Organic Matter [J]. Analytical Chemistry, 2023, 95 (5): 2796-2803) employed in the present invention enables efficient and accurate matching of halogenated organic compounds.

In molecular formula matching, molecular screening, molecular information summarization, and statistical analysis of sample data for FT-ICR MS detection raw down-machine data using FTMSAnalysis platform, the union of the molecules matched by the HLB column and the PPL column for the same sample is the total halo-dissolvable organic substances in the sample, and the halo-dissolvable organic molecules with the same molecular formula are calculated only once, and then are analyzed and visualized by Sankey diagrams for the type, composition distribution and property of the halo-dissolvable organic substances in the samples.

The present invention has the following advantages:
(1) By simultaneous solid phase extracting one sample using a combined solid phase extraction method of HLB and PPL solid phase extraction, and detection of HOCs in environmental samples using non-targeted ultra-high resolution FT-ICR MS technique, enables more thorough access to molecular information of HOCs in the sample, particularly dissolvable halogenated organic substances containing Cl, Br molecules;
(2) Acidifying the sample prior to solid phase extraction by using formic acid in place of conventionally used HCl can effectively avoid acidifying the sample with conventionally used HCl that may react with certain components in the sample to generate new chlorine containing organic substances; in addition to acidification of the sample using formic acid, it is also possible to screen out large molecular weight humus in the sample in the solid phase extraction stage, thereby efficiently enriching small molecular weight organic substances, the HOCs being small molecular weight, which can avoid large molecular weight humus interfering with subsequent FT-ICR MS detection of the HOCs.
(3) It is important to demonstrate more clearly and comprehensively molecular type, molecular properties and composition distribution characteristics of halo-dissolvable organic molecules in sample and in water bodies by analysis using molecular information of HOCs in sample of Sankey diagrams.

### DESCRIPTION OF THE FIGURES

FIG. 1 shows a Sankey diagram of molecular information and properties of halogenated organic substances in groundwater samples in Example 1;

FIG. 2 is a graph showing the effect of the removal of large molecular weight humic substances and enrichment of middle and small molecular weight organic substances in groundwater samples during the solid phase extraction stage in Example 1;

FIG. 3 shows a Sankey diagram of molecular information and properties of halogenated organic substances in landfill leachate stock samples of example 2;

FIG. 4 is a graph showing the effect of the removal of large molecular weight humic substances and enrichment of middle and small molecular weight organic substances in landfill leachate stock samples during the solid phase extraction stage in Example 2.

### DETAILED DESCRIPTION OF INVENTION

The present invention will now be described in further detail with reference to the accompanying drawings and preferred embodiments. From these descriptions, the characteristics and advantages of the present invention will become more clearly apparent.

### EXAMPLE 1

A sample of groundwater contaminated with leachate is collected from a living landfill, stored at 4° C., and transported to laboratory within one week, and passed through 0.45 μm aqueous filter membrane. The dissolvable organic carbon (DOC) of the sample is measured as 24.86 mg/L.

Calculating as 30-50% retention of solid phase extraction column when the volume of the sample is 18-22 mL, DOC adsorbed by solid phase extraction column is eluted down to 1 mL and the value of DOC is between 90-110 mg/L.

Two samples of water, each 20 mL in volume, are taken and the pH of which is adjusted to 2.0 with 98% formic acid solution. An HLB solid phase extraction column and a PPL solid phase extraction column are mounted on a solid phase extraction device, respectively. The HLB solid phase extraction column is rinsed and activated sequentially with 12 mL methanol and 12 mL 0.01 mol/L formic acid solution, and the PPL solid phase extraction column is rinsed and activated sequentially with 12 mL methanol and 12 mL 0.01 mol/L formic acid solution. The samples of water are slowly added respectively to the HLB solid phase extraction column and the PPL solid phase extraction column at a flow rate of 5 mL min⁻¹. After the samples of water flow out, the HLB solid phase extraction column and the PPL solid phase extraction column are washed respectively with 18 mL 0.01 mol/L formic acid, and are blown and dried with nitrogen, and then are eluted with 12 mL methanol separately. The eluents are collected and blown with nitrogen until methanol is fully volatilized, to obtain enriched samples from the HLB and PPL solid phase extraction columns, respectively. The samples are dissolved with 1 mL methanol and are subjected to FT-ICR MS detection, respectively, and then molecular formulas are matched with FTMSAnalysis platform.

FT-ICR MS measurements are performed using SolariX type FT-ICR MS from Bruker Company, and the ion source is electrospray ion source (ESI) in negative ion mode, with detection parameters as follows: injection mode is continuous injection, injection speed is 120 μL/h, capillary inlet voltage is 4.0 kV, ion accumulation time is 0.06 s, acquisition mass range is from 100 to 1600 Da, sample point number is 4 M 32-bit data, time domain signal superposition is 300 times to improve signal-to-noise ratio.

The final matched molecular formulas of halogenated organic substances in groundwater polluted with the leachate obtained from HLB and PPL solid phase extraction columns are shown in Tables 1 and 2.

TABLE 1
Halogenated organic molecules in groundwater polluted with the
leachate obtained from HLB solid phase extraction column

| | | Molecular | |
|---|---|---|---|
| Molecular | Molecular | Element | Molecular |
| Type | Formula | Composition | Property |
| Monochloro- | C7H7O3S1Cl1 | CHOSCl1 | lignin |
| dissolvable organic | | | |
| substance | | | |
| Dichloro-dissolvable | C8H8O6Cl2 | CHOCl2 | tannin |
| organic substance | C15H14O3Cl2 | CHOCl2 | lignin |
| | C10H10O8Cl2 | CHOCl2 | tannin |
| Trichloro- | C12H17O9Cl3 | CHOCl3 | tannin |
| dissolvable organic | | | |
| substance | | | |
| Pentachloro- | C9H5O4Cl5 | CHOCl5 | condensed aromatic |
| dissolvable organic | | | hydrocarbon |
| substance | | | |

TABLE 2
Halogenated organic molecules in groundwater polluted with
leachate obtained from PPL solid phase extraction column

| | | Molecular | |
|---|---|---|---|
| Molecular | Molecular | Element | Molecular |
| Type | Formula | Composition | Property |
| Monochloro- | C7H7O3S1Cl1 | CHOSCl1 | lignin |
| dissolvable | C16H11O6N2S1Cl1 | CHONSCl1 | condensed aromatic |
| organic | | | hydrocarbon |
| substance | C7H8O3N1S1Cl1 | CHONSCl1 | lignin |
| | C8H8O4N1S1Cl1 | CHONSCl1 | lignin |
| Dichloro- | C8H8O6Cl2 | CHOCl2 | tannin |
| dissolvable | C15H14O3Cl2 | CHOCl2 | lignin |
| organic | | | |
| substance | | | |
| Pentachloro- | C9H5O4Cl5 | CHOCl5 | condensed aromatic |
| dissolvable | | | hydrocarbon |
| organic | | | |
| substance | | | |
| Monobromine- | C14H21O6Br1 | CHOBr1 | lignin |
| dissolvable | C15H21O6Br1 | CHOBr1 | lignin |
| organic | | | |
| substance | | | |
| Dibromine- | C9H6O6Br2 | CHOBr2 | condensed aromatic |
| dissolvable | | | hydrocarbon |
| organic | C26H52O8Br2 | CHOBr2 | nitrogen-free saturated |
| substance | | | hydrocarbon |
| | C25H50O9Br2 | CHOBr2 | nitrogen-free saturated |
| | | | hydrocarbon |
| | C27H54O8Br2 | CHOBr2 | nitrogen-free saturated |
| | | | hydrocarbon |
| | C27H52O9Br2 | CHOBr2 | nitrogen-free saturated |
| | | | hydrocarbon |
| | C28H56O8Br2 | CHOBr2 | nitrogen-free saturated |
| | | | hydrocarbon |
| | C27H54O9Br2 | CHOBr2 | nitrogen-free saturated |
| | | | hydrocarbon |
| | C29H58O8Br2 | CHOBr2 | nitrogen-free saturated |
| | | | hydrocarbon |
| | C27H52O10Br2 | CHOBr2 | nitrogen-free saturated |
| | | | hydrocarbon |
| | C28H56O9Br2 | CHOBr2 | nitrogen-free saturated |
| | | | hydrocarbon |
| | C30H60O8Br2 | CHOBr2 | nitrogen-free saturated |
| | | | hydrocarbon |
| | C30H58O9Br2 | CHOBr2 | nitrogen-free saturated |
| | | | hydrocarbon |
| Monochloro- | C9H6O6Cl1Br1 | CHOCl1Br1 | condensed aromatic |
| monobromine | | | hydrocarbon |
| dissolvable | C30H48O11Cl1Br1 | CHOCl1Br1 | nitrogen-free saturated |
| organic | | | hydrocarbon |
| substance | C31H52O10Cl1Br1 | CHOCl1Br1 | nitrogen-free saturated |
| | | | hydrocarbon |
| | C30H50O11Cl1Br1 | CHOCl1Br1 | nitrogen-free saturated |
| | | | hydrocarbon |
| | C32H54O10Cl1Br1 | CHOCl1Br1 | nitrogen-free saturated |
| | | | hydrocarbon |
| | C34H54O12Cl1Br1 | CHOCl1Br1 | nitrogen-free saturated |
| | | | hydrocarbon |

The results show that, HLB solid phase extraction column is able to enrich more chlorinated dissolvable organic substances, which detected more C10H10O8C12 and C12H17O9C13 than PPL column, while PPL solid phase extraction column is able to enrich more bromine-containing halogenated organic substances, and molecular formulas from both are merged and the results are shown in Table 3.

TABLE 3
All halogenated organic molecules in groundwater polluted by the leachate

| | | Molecular | |
|---|---|---|---|
| Molecular | Molecular | Element | Molecular |
| Type | Formula | Composition | Property |
| Monochloro- | C7H7O3S1Cl1 | CHOSCl1 | lignin |
| dissolvable | C16H11O6N2S1Cl1 | CHONSCl1 | condensed aromatic |
| organic | | | hydrocarbon |
| substance | C7H8O3N1S1Cl1 | CHONSCl1 | lignin |
| | C8H8O4N1S1Cl1 | CHONSCl1 | lignin |
| Dichloro- | C8H8O6Cl2 | CHOCl2 | tannin |
| dissolvable | C15H14O3Cl2 | CHOCl2 | lignin |
| organic | C10H10O8Cl2 | CHOCl2 | tannin |
| substance | | | |
| Trichloro- | C12H17O9Cl3 | CHOCl3 | tannin |
| dissolvable | | | |
| organic | | | |
| substance | | | |
| Pentachloro- | C9H5O4Cl5 | CHOCl5 | condensed aromatic |
| dissolvable | | | hydrocarbon |
| organic | | | |
| substance | | | |
| Monobromine | C14H21O6Br1 | CHOBr1 | lignin |
| dissolvable | C15H21O6Br1 | CHOBr1 | lignin |
| organic | | | |
| substance | | | |
| Dibromine- | C9H6O6Br2 | CHOBr2 | condensed aromatic |
| dissolvable | | | hydrocarbon |
| organic | C26H52O8Br2 | CHOBr2 | nitrogen-free saturated |
| substance | | | hydrocarbon |
| | C25H50O9Br2 | CHOBr2 | nitrogen-free saturated |
| | | | hydrocarbon |
| | C27H54O8Br2 | CHOBr2 | nitrogen-free saturated |
| | | | hydrocarbon |
| | C27H52O9Br2 | CHOBr2 | nitrogen-free saturated |
| | | | hydrocarbon |
| | C28H56O8Br2 | CHOBr2 | nitrogen-free saturated |
| | | | hydrocarbon |
| | C27H54O9Br2 | CHOBr2 | nitrogen-free saturated |
| | | | hydrocarbon |
| | C29H58O8Br2 | CHOBr2 | nitrogen-free saturated |
| | | | hydrocarbon |
| | C27H52O10Br2 | CHOBr2 | nitrogen-free saturated |
| | | | hydrocarbon |
| | C28H56O9Br2 | CHOBr2 | nitrogen-free saturated |
| | | | hydrocarbon |
| | C30H60O8Br2 | CHOBr2 | nitrogen-free saturated |
| | | | hydrocarbon |
| | C30H58O9Br2 | CHOBr2 | nitrogen-free saturated |
| | | | hydrocarbon |
| Monohloro- | C9H6O6Cl1Br1 | CHOCl1Br1 | condensed aromatic |
| monobromine | | | hydrocarbon |
| dissolvable | C30H48O11Cl1Br1 | CHOCl1Br1 | nitrogen-free saturated |
| organic | | | hydrocarbon |
| substance | C31H52O10Cl1Br1 | CHOCl1Br1 | nitrogen-free saturated |
| | | | hydrocarbon |
| | C30H50O11Cl1Br1 | CHOCl1Br1 | nitrogen-free saturated |
| | | | hydrocarbon |
| | C32H54O10Cl1Br1 | CHOCl1Br1 | nitrogen-free saturated |
| | | | hydrocarbon |
| | C34H54O12Cl1Br1 | CHOCl1Br1 | nitrogen-free saturated |
| | | | hydrocarbon |

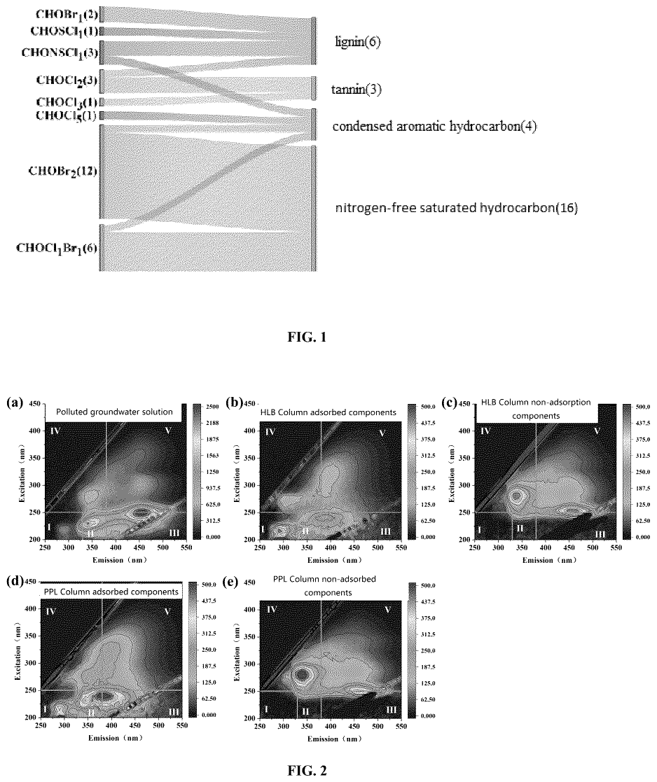

For clearer and more comprehensive representation of molecular information and properties of halogenated organic substances in groundwater polluted with leachate, the molecular information and properties of the halogenated organic substances are represented using the Sankey plotting function in Origin software, based on the molecular information in Table 3, resulting in a Sankey diagram of the molecular information and properties of halogenated organic substances in the sample as shown in FIG. 1.

To further validate above detection method, fluorescence spectroscopy techniques are used to characterize the stock solution of the ground water polluted with the leachate, the eluent from solid phase extraction column (column adsorbed DOM, i.e. DOM that can be subsequently detected by FT-ICR MS) and the stream from solid phase extraction column (column non-adsorbed DOM, i.e. DOM that has not been extracted, that cannot be detected by FT-ICR MS), respectively, and the three-dimensional fluorescence spectrogram is shown in FIG. 2. A three-dimensional fluorescence spectrogram is a matrix spectrum characterized by three-dimensional coordinates of excitation wavelength (y-axis)-emission wavelength (x-axis)-fluorescence intensity (z-axis). Dissolvable fluorescent organics with different nature are located at different positions, and the fluorescence intensity then represents the high and low concentration of dissolved fluorescent organics. Fluorescence Region Integration method divides the three-dimensional fluorescence spectrogram into different regions, and each region represents a portion of dissolved organic substances, including tyrosine and tyrosine-like (region I), tryptophan and tryptophan-like (region II), fulvic acid and fulvic acid like (region III), dissolvable microbial products (region IV) and humic acids (region V). In general, dissolvable organics with larger excitation and emission wavelengths have larger molecular weights, so dissolvable microbial products, humic acids represented by regions IV, V have larger molecular weights, while DOMs represented by regions I, II and III have middle and small molecular weights.

FIG. 2 shows that, both columns have better adsorption effect on DOMs with middle and small molecular weights in groundwater polluted with leachate, thus DOMs with middle and small molecular weights can be enriched efficiently by the method including formic acidification, PPL and HLB combined solid phase extraction and elution with methanol, and DOMs with large molecular weights (soluble microbial products, humic acid) in the sample of water can be effectively screened out. As HOCs belong to small molecular compounds, with molecular weights typically between tens and hundreds of Daltons, the method of the invention enables directed, efficient enrichment of HOCs in groundwater polluted with leachate, and can effectively screen out dissolvable microbial products, humic acid with large molecular weight in the sample of water, to avoid their interference with HOCs testing in the subsequent mass spectrometric testing.

### EXAMPLE 2

The waste leachate stock solution from the living landfill in Example 1 is collected, stored at 4° C. and transported to the laboratory within one week. The sample of the leachate is passed through 0.45 μm filter membrane and then the DOC of the sample is measured to be 875 mg/L, while as the DOC of the groundwater polluted with the leachate is 24.86 mg/L (see Example 1). The leachate is diluted with deionized water to the DOC of 24.86 mg/L, while 10 mL of the leachate stock solution is taken into a 500 mL beaker, and then is diluted with 342 mL of deionized water and is stored ready for use.

Calculating as 30-50% retention of solid phase extraction column when the volume of the sample is 18-22 mL, DOC adsorbed by solid phase extraction column is eluted down to 1 mL and the value of DOC is between 90-110 mg/L.

Two samples of diluted water (each 20 mL in volume) are taken, and the pH of which is adjusted to 2.0 with 98% formic acid solution. An HLB solid phase extraction column and a PPL solid phase extraction column are mounted on a solid phase extraction device, respectively. The HLB solid phase extraction column is rinsed and activated sequentially with 12 mL methanol and 12 mL 0.01 mol/L formic acid solution, and the PPL solid phase extraction column is rinsed and activated sequentially with 12 mL methanol and 12 mL 0.01 mol/L formic acid solution. The samples of water are slowly added respectively to the HLB solid phase extraction column and the PPL solid phase extraction column at a flow rate of 5 mL min⁻¹. After the samples of water flow out, the HLB solid phase extraction column and the PPL solid phase extraction column are washed respectively with 18 mL 0.01 mol/L formic acid, and are blown and dried with nitrogen, and then are eluted with 12 mL methanol separately. The eluents are collected and blown with nitrogen until methanol is fully volatilized, to obtain enriched samples from the HLB and PPL solid phase extraction columns, respectively. The samples are dissolved with 1 mL methanol and are subjected to FT-ICR MS detection, respectively, and then molecular formulas are matched with FTMSAnalysis platform.

The final matched molecular formulas of halogenated organic substances in samples of leachate stock solution obtained from HLB and PPL solid phase extraction columns are shown in Tables 4 and 5.

TABLE 4
Halogenated organic molecules in leachate stock solution
obtained from HLB solid phase extraction column

| Molecular | Molecular | Molecular Element | Molecular |
|---|---|---|---|
| Type | Formula | Composition | Property |
| Monochloro- | C7H7O3S1Cl1 | CHOSCl1 | lignin |
| dissolvable | C7H8O3N1S1Cl1 | CHONSCl1 | lignin |
| organic | C7H7O4S1Cl1 | CHOSCl1 | lignin |
| substance | C8H10O3N1S1Cl1 | CHONSCl1 | lignin |
| | C8H8O4N1S1Cl1 | CHONSCl1 | lignin |
| | C8H7O5S1Cl1 | CHOSCl1 | lignin |
| | C8H9O3S2Cl1 | CHOSCl1 | lignin |
| | C9H10O5N1S1Cl1 | CHONSCl1 | lignin |
| | C10H12O5N1S1Cl1 | CHONSCl1 | lignin |
| | C12H15O4N2S1Cl1 | CHONSCl1 | lignin |
| | C14H14O5N1S1Cl1 | CHONSCl1 | lignin |
| | C20H18O5N3Cl1 | CHONCl1 | lignin |
| | C20H17O6N2Cl1 | CHONCl1 | lignin |
| | C20H19O6N2Cl1 | CHONCl1 | lignin |
| | C21H20O5N3Cl1 | CHONCl1 | lignin |
| | C21H23O6N2Cl1 | CHONCl1 | lignin |
| | C22H25O6N2Cl1 | CHONCl1 | lignin |
| | C22H20O6N3Cl1 | CHONCl1 | lignin |
| Dichloro- | C8H8O6Cl2 | CHOCl2 | tannin |
| dissolvable | C15H14O3Cl2 | CHOCl2 | lignin |
| organic | | | |
| substance | | | |
| Pentachloro- | C9H5O4Cl5 | CHOCl5 | condensed aromatic |
| dissolvable | | | hydrocarbon |
| organic | | | |
| substance | | | |
| Monobromine- | C21H21O1Br1 | CHOBr1 | unsaturated hydrocarbon |
| dissolvable | C22H23O1Br1 | CHOBr1 | unsaturated hydrocarbon |
| organic | C22H25O1Br1 | CHOBr1 | unsaturated hydrocarbon |
| substance | C23H23O2Br1 | CHOBr1 | unsaturated hydrocarbon |

TABLE 5
Halogenated organic molecules in leachate stock solution
finally obtained from PPL solid phase extraction column

| Molecular | Molecular | Molecular Element | Molecular |
|---|---|---|---|
| Type | Formula | Composition | Property |
| Monochloro- | C7H6O6N1S1Cl1 | CHONSCl1 | tannin |
| dissolvable | C7H7O3S1Cl1 | CHOSCl1 | lignin |
| organic | C7H8O3N1S1Cl1 | CHONSCl1 | lignin |
| substance | C7H7O4S1Cl1 | CHOSCl1 | lignin |
| | C8H10O3N1S1Cl1 | CHONSCl1 | lignin |
| | C8H8O4N1S1Cl1 | CHONSCl1 | lignin |
| | C8H7O5S1Cl1 | CHOSCl1 | lignin |
| | C8H9O3S2Cl1 | CHOSCl1 | lignin |
| | C9H10O5N1S1Cl1 | CHONSCl1 | lignin |
| | C10H12O5N1S1Cl1 | CHONSCl1 | lignin |
| | C11H16O3N1S1Cl1 | CHONSCl1 | lignin |
| | C16H13O3N2S1Cl1 | CHONSCl1 | lignin |
| | C16H11O6N2S1Cl1 | CHONSCl1 | condensed aromatic |
| | | | hydrocarbon |
| | C17H21O4N2Cl1 | CHONCl1 | lignin |
| | C19H19O5N2Cl1 | CHONCl1 | lignin |
| | C20H20O4N3Cl1 | CHONCl1 | lignin |
| | C20H18O5N3Cl1 | CHONCl1 | lignin |
| | C20H19O6N2Cl1 | CHONCl1 | lignin |
| Dichloro- | C8H8O6Cl2 | CHOCl2 | tannin |
| dissolvable | C15H14O3Cl2 | CHOCl2 | lignin |
| organic | | | |
| substance | | | |
| Pentachloro- | C9H5O4Cl5 | CHOCl5 | condensed aromatic |
| dissolvable | | | hydrocarbon |
| organic | | | |
| substance | | | |
| Monobromine- | C21H21O1Br1 | CHOBr1 | unsaturated hydrocarbon |
| dissolvable | C22H23O1Br1 | CHOBr1 | unsaturated hydrocarbon |
| organic | C22H25O1Br1 | CHOBr1 | unsaturated hydrocarbon |
| substance | C23H23O2Br1 | CHOBr1 | unsaturated hydrocarbon |
| | C24H25O2Br1 | CHOBr1 | unsaturated hydrocarbon |
| | C26H31O2Br1 | CHOBr1 | unsaturated hydrocarbon |
| Dibromine | C7H4O3Br2 | CHOBr2 | condensed aromatic |
| dissolvable | | | hydrocarbon |
| organic | | | |
| substance | | | |
| Monochloro- | C12H24O7Cl1Br1 | CHOCl1Br1 | amino sugar |
| monobromide | | | |
| dissolvable | | | |
| organic | | | |
| substance | | | |

From Tables 4 and 5, it can be seen that, halo-dissolvable organic substances finally obtained from HLB and PPL columns differs significantly between chloro-dissolvable organic substance and bromo-dissolvable organic substance, and molecular formulas from both are merged and the results are shown in Table 6.

TABLE 6
All halogenated organic molecules in the leachate

| | | Molecular | |
|---|---|---|---|
| Molecular | Molecular | Element | Molecular |
| Type | Formula | Composition | Property |
| Monochloro- | C7H6O6N1S1Cl1 | CHONSCl1 | tannin |
| dissolvable | C7H7O3S1Cl1 | CHOSCl1 | lignin |
| organic | C7H8O3N1S1Cl1 | CHONSCl1 | lignin |
| substance | C7H7O4S1Cl1 | CHOSCl1 | lignin |
| | C8H10O3N1S1Cl1 | CHONSCl1 | lignin |
| | C8H8O4N1S1Cl1 | CHONSCl1 | lignin |
| | C8H7O5S1Cl1 | CHOSCl1 | lignin |
| | C8H9O3S2Cl1 | CHOSCl1 | lignin |
| | C9H10O5N1S1Cl1 | CHONSCl1 | lignin |
| | C10H12O5N1S1Cl1 | CHONSCl1 | lignin |
| | C11H16O3N1S1Cl1 | CHONSCl1 | lignin |
| | C12H15O4N2S1Cl1 | CHONSCl1 | lignin |
| | C14H14O5N1S1Cl1 | CHONSCl1 | lignin |
| | C16H13O3N2S1Cl1 | CHONSCl1 | lignin |
| | C16H11O6N2S1Cl1 | CHONSCl1 | condensed |
| | | | aromatic |
| | | | hydrocarbon |
| | C17H21O4N2Cl1 | CHONCl1 | lignin |
| | C19H19O5N2Cl1 | CHONCl1 | lignin |
| | C20H17O6N2Cl1 | CHONCl1 | lignin |
| | C20H18O5N3Cl1 | CHONCl1 | lignin |
| | C20H19O6N2Cl1 | CHONCl1 | lignin |
| | C20H20O4N3Cl1 | CHONCl1 | lignin |
| | C21H20O5N3Cl1 | CHONCl1 | lignin |
| | C21H23O6N2Cl1 | CHONCl1 | lignin |
| | C22H25O6N2Cl1 | CHONCl1 | lignin |
| | C22H20O6N3Cl1 | CHONCl1 | lignin |
| Dichloro- | C8H8O6Cl2 | CHOCl2 | tannin |
| dissolvable | C15H14O3Cl2 | CHOCl2 | lignin |
| organic | | | |
| substance | | | |
| Pentachloro- | C9H5O4Cl5 | CHOCl5 | condensed |
| dissolvable | | | aromatic |
| organic | | | hydrocarbon |
| substance | | | |
| Monobromine- | C21H21O1Br1 | CHOBr1 | unsaturated |
| dissolvable | | | hydrocarbon |
| organic | C22H23O1Br1 | CHOBr1 | unsaturated |
| substance | | | hydrocarbon |
| | C22H25O1Br1 | CHOBr1 | unsaturated |
| | | | hydrocarbon |
| | C23H23O2Br1 | CHOBr1 | unsaturated |
| | | | hydrocarbon |
| | C24H25O2Br1 | CHOBr1 | unsaturated |
| | | | hydrocarbon |
| | C26H31O2Br1 | CHOBr1 | unsaturated |
| | | | hydrocarbon |
| Dibromine- | C7H4O3Br2 | CHOBr2 | condensed |
| dissolvable | | | aromatic |
| organic | | | hydrocarbon |
| substance | | | |
| Monochloro- | C12H24O7Cl1Br1 | CHOCl1Br1 | amino sugar |
| monobromide | | | |
| dissolvable | | | |
| organic | | | |
| substance | | | |

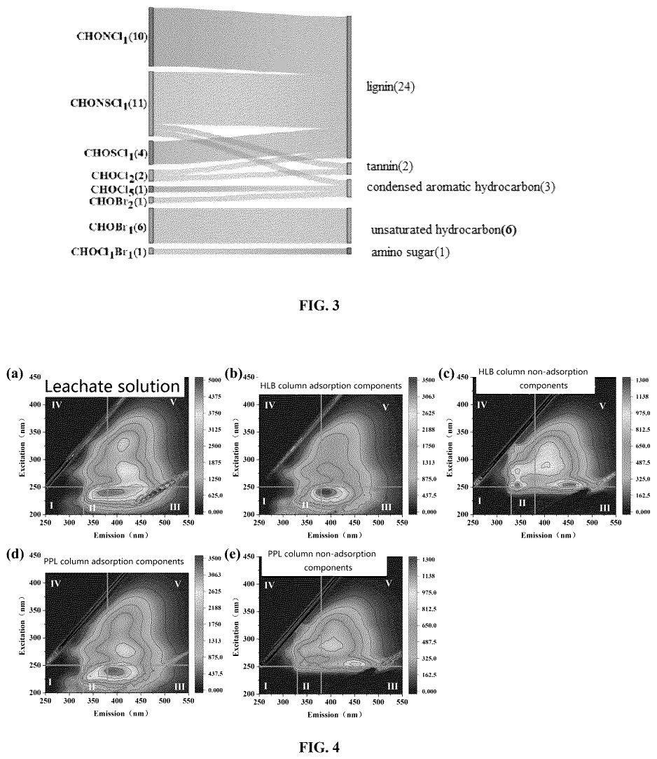

For clearer and more comprehensive representation of the molecular information and properties of halogenated organic substances in the leachate, the molecular information and properties of the halogenated organic substances in the leachate are expressed using the Sankey plotting function in Origin software, based on the molecular information in Table 6, which finally results in a Sankey diagram of the molecular information and properties of halogenated organic substances in the leachate as shown in FIG. 3.

To further validate the validity of above detection method, fluorescence spectroscopy techniques are used to characterize separately the stock solution of the leachate (50-folds dilution), the eluent from solid phase extraction column (column adsorbed DOM, i.e. DOM that can be subsequently detected by FT-ICR MS) and the stream from solid phase extraction column (column non-adsorbed DOM, i.e. DOM that has not been extracted, that cannot be detected by FT-ICR MS), as shown in FIG. 4, in which, both columns have better adsorption effect on DOMs with middle and small molecular weights in the leachate represented by regions I, II and III, and DOMs with large molecular weights (dissolvable microbial products, humic acid) in the sample can be effectively screened out, thus the method of the invention enables directed, efficient enrichment of HOCs in the leachate and can effectively screen out soluble microbial products, humic acid with large molecular weight in the leachate, to avoid their interference with the testing of HOCs at the subsequent stage of mass spectrometry testing.

The present invention has been described in detail above with reference to preferred embodiments and exemplary examples. It is nevertheless stated that These detailed description and examples are only illustrative explanations of the invention, no limitation is to be placed upon the scope of protection of the invention. Various modifications, substitutions or modifications may be made to the technical disclosure and its embodiments without departing from the spirit and scope of protection of the invention, all falling within the scope of protection of the invention. The scope of protection of the invention shall be subject to the appended claims.

What is claimed is:

1. A method for detecting halogenated organic substances in landfill leachate and polluted water bodies thereof, by combined solid phase extraction method comprising HLB solid phase extraction and PPL solid phase extraction, the aqueous samples are enriched by solid phase extraction using an HLB solid phase extraction column and a PPL solid phase extraction column, respectively, the enriched samples are then subjected to FT-ICR MS detection, and two detection data of FT-ICR MS are subjected to molecular formula matching of organic molecules, molecular screening, molecular information summarization, and/or statistical analysis of the data of samples, respectively, and the two are combined to obtain all dissolvable halogenated organic substances in the samples of water.

2. The method according to claim 1, wherein fresh leachate stocks collected from living landfills and samples of groundwater upstream and downstream near the landfills are transported within one week after collection to a laboratory for preservation and pre-treatment.

3. The method according to claim 1, wherein collected aqueous sample is passed through a filter membrane, preferably 0.45 μm aqueous filter membrane, to sufficiently remove poorly dissolvable organic substances, such as suspended matters, and the concentration of dissolvable organic carbon (DOC) is measured, and then the volume required for solid phase extraction is determined based on the concentration of DOC.

4. The method according to claim 3, wherein FT-ICR MS detection is performed when the value of DOC is between 90-110 mg/L while the DOC adsorbed by the solid phase extraction columns is enriched to 1 mL after elution.

5. The method according to claim 3, wherein respective DOC of the landfill leachate stock solution, and upstream and downstream groundwater is measured after passing through filter membrane, whereby the lowest DOC of one water sample is normalized, and other water sample is diluted until its DOC equals the lowest DOC.

6. The method according to claim 1, wherein the volume of water sample required for solid phase extraction is determined based on enrichment of DOC of between 30% and 50% by the solid phase extraction column.

7. The method according to claim 6, wherein, two samples of filtered water are taken separately, and the pH of the samples is adjusted to a predetermined value with formic acid solution; an HLB solid phase extraction column and a PPL solid phase extraction column are mounted on a solid phase extraction device, respectively, and each solid phase extraction column is rinsed and activated sequentially with methanol and formic acid solution, and then the water samples are slowly added to the HLB solid phase extraction column and the PPL solid phase extraction column, respectively at a flow rate, to enrich halogenated organic compounds; and after the water samples flow out, formic acid is added to wash the HLB solid phase extraction Column and the PPL solid phase extraction column, respectively, to remove salts, and then the columns are dried by nitrogen blowing and are eluted with methanol to collect the eluents separately, which are blown with nitrogen until methanol is fully volatilized, to yield enriched samples from HLB and PPL solid phase extraction columns, respectively, for subsequent FT-ICR MS detection.

8. The method according to claim 1, wherein molecular formula matching of organic molecules is performed on two FT-ICR MS detection data by dissolvable organic substances ultra-resolution mass spectrometry matching platform FTMSAnalysis, which is developed based on Gaussian distribution alignment algorithm.

9. The method according to claim 8, wherein the union of the molecules matched by the HLB column and the PPL column for the same sample is the total halo-dissolvable organic substances in the sample; and the halo-dissolvable organic molecules with the same molecular formula are calculated only once, and then are analyzed and visualized by Sankey diagrams for the type, composition distribution and property of the halo-dissolvable organic substances in the samples.
